(19) United States
(12) Patent Application Publication
(10) Pub. No.: US 2006/0014213 A1
(43) Pub. Date: Jan. 19, 2006

(54) Methods for rapid screening of mad cow disease and other transmissible spongiform encephalopathies

(75) Inventors: Cha Min Tang, Wayne, PA (US); Robert G. Rohwer, Ellicot City, MD (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignees: Department of Veterans Affairs; University of Maryland

(21) Appl. No.: 11/108,118

(22) Filed: Apr. 18, 2005

Related U.S. Application Data
(60) Provisional application No. 60/563,538, filed on Apr. 19, 2004.

(51) Int. Cl. G01N 33/53

(52) U.S. Cl. 435/7.1

(57) ABSTRACT

Methods for diagnosing altered neuropathology in an animal are disclosed, wherein said methods comprise imaging brain, spinal cord, or other neural tissue of the animal, analyzing the appearance of the tissue, and determining whether the appearance of the tissue is altered relative to corresponding unaltered tissue. Also disclosed are methods for diagnosing spongiform encephalopathies in an animal, wherein said methods comprise imaging brain, spinal cord, or other neural tissues of the animal, analyzing the appearance of vacuoles in the tissue, and determining whether the appearance of the vacuoles in the tissue is altered relative to corresponding spongiform encephalopathy-free tissue. Also disclosed are automated methods for diagnosing altered neuropathy and spongiform encephalopathies.

This work was supported by NINDS Grant No. NS44627, and therefore the government may have certain rights to the invention.

### FIELD OF THE INVENTION

The present invention relates to methods of diagnosing diseases involving altered neuropathology. Included are methods for rapid screening of mad cow disease and other transmissible spongiform encephalopathies. These methods utilize visualization techniques such as optical coherence tomography (OCT).

### BACKGROUND OF THE INVENTION

Mad Cow disease (also know as BSE, bovine spongiform encephalopathy) has had an enormous negative impact on the economies Great Britain, Canada, and now the US. The definitive means for documenting transmissible spongiform encephalopathies (TSE) such as Creutzfield-Jakob disease (CJD) in humans, bovine spongiform encephalopathy (BSE or Mad Cow disease), scrapie in sheep, and chronic wasting disease (CWD) in deer and elk is to transmit disease to another animal. But practical diagnosis is generally made based on the presence of characteristic spongiform changes in the brain and/or the presence of certain protease resistant proteins (PrP) (Moynagh, J., et al., (1999) The evaluation of Tests for the Diagnosis of Transmissible Spongiform Encephalopathy in Bovines, European Commission, Directorate B—Scientific Health Opinions). Current tests mainly utilize ELISA or Western blots to detect the protease resistant PrP. These tests require biopsy of tissue and typically take hours to complete. These tests are not optimal for rapid screening of large numbers of animals. Furthermore, they are not well suited for in vivo testing. The present invention provides a needed simpler and faster screening test.

### SUMMARY OF THE INVENTION

The present invention relates to a method of diagnosis of a spongiform encephalopathy. This method includes imaging the brain, spinal cord, or other neural tissue of an animal, analyzing the vacuole appearance, determining if vacuole is altered, as compared with the neuropathology of an animal known to lack spongiform encephalopathy. Vacuoles which are widely distributed, demonstrate a high degree of back scattering, or are large indicate that the animal has or had a spongiform encephalopathy. The imaging may be done with a catheter-based OCT probe with a rigid cannula. The spongiform encephalopathy may be CJD, BSE, TSE, CWD or scrapie, for example.

The present invention relates to the combination of the above method with a different method of diagnosing a spongiform encephalopathy.

The present invention relates to a method of diagnosis of any disease involving altered neuropathology. This method includes imaging the brain, spinal cord, or other neural tissue of an animal. The neuropathology is subsequently analyzed and compared to the neuropathology of particular disease states. Neuropathology similar to a particular disease is an indication that the subject has the particular disease.

### BRIEF DESCRIPTION OF DRAWINGS

The objects and advantages of the invention will be understood by reading the following detailed description in conjunction with the drawings in which:

FIG. 1 illustrates the OCT imaging of brain tissue from the parahippocampal cortex of a human who died of Creutzfield-Jakob disease (CJD).

FIG. 2 illustrates the OCT imaging of the stratum brain tissue of a hamster infected with scrapie.

FIG. 3 illustrates the OCT imaging of the olfactory bulb of a mouse brain infected with BSE.

### DETAILED DESCRIPTION OF ILLUSTRATIVE EMBODIMENTS

Current methods of diagnosing transmissible spongiform encephalopathies (TSE) utilize biochemical methods such as ELISA or Western blots. These tests require biopsy of tissue, typically take hours to complete, are not optimal for rapid screening of large numbers of animals, and are not optimal for in vivo testing. Particularly considered public health concerns related to mad cow disease, improved methods of detection, screening and diagnosis are needed. The present invention provides methods of diagnosing TSE, including, but not limited to, CWD, CJD, BSE, and scrapie. The methods of the present invention provide a higher sample throughput than current methods, in part, because of the ability to test live or dead animals. The subject invention is faster and simpler than prior art methods. Another advantage of the present invention is use in screening large numbers of animals.

The present invention is useful in the diagnosis of any diseases which alter neuropathology (e.g. the pathology of the nervous system). In particular, the present invention is useful in the diagnosis of any diseases which alter vacuoles or, alternatively, form plaques in a tissue. For example, the present invention teaches the diagnosis of transmissible spongiform encephalopathies (TSE) such as, but not limited to, bovine spongiform encephalopathy (BSE or Mad Cow disease), scrapie in sheep, and chronic wasting disease (CWD) of deer. In an alternative embodiment, the subject invention is used to identify human patients with Creutzfield-Jakob disease (CJD) In a further embodiment, the present invention provides a method of distinguishing sporadic from variant and/or familial forms of the disease. It is contemplated that the methods described herein are further useful for the diagnosis of Gerstmann-Streussler-Sheinker Disease (GSS), fatal familial insomnia (FFI), hereditary Icelandic syndrome, senility and multiple myeloma, for example.

Method of Diagnosis in Dead Animal

Included are methods of diagnosis of a dead animal. Tissue of slaughtered animals is provided. The tissue may be any body tissue known to be vulnerable to the pathological effect of the disease, such as, for example, neural tissue, including, but not limited to brain and spinal cord tissues. Tissue deep in the brain is also contemplated. For example, the tissue is accessed by the use of a probe. More specifically, a needle type probe may be inserted directly through thin regions of the skull. Alternatively, the probe may be inserted through the roof of the orbit below the eye brow to sample the frontal cortex.

The tissue is imaged. For example, a radial scan is performed to image the brain. The probe may be advanced to sample a volume of tissue. The data may be analyzed by the operator in real time. Alternatively, the data may be stored for off-line processing. A skilled artisan is aware of methods well known in the art for processing such data regardless of whether the processing is performed at the time of data acquisition. It is contemplated that software may be developed to automatically identify, measure, and count the number of vacuole per volume of tissue sampled. For example, the index of refraction of the vacuole may also be determined based on the amplitude of reflected light using methods well known in the art. These data will be analyzed using statistical criteria that define the likelihood of TSE in specific brain regions, the animal, and stage of the disease.

Imaging Techniques

Various imaging techniques are useful in the methods of the present invention. Exemplary techniques are described in International Application Number PCT/US2003/028352, which is hereby incorporated by reference herein. In an exemplary embodiment, imaging is performed using a needle-type probe. Other, non-limiting, examples of imaging techniques are contemplated and include, for example, contact but non-penetrating imaging, and non-contact imaging.

In the contact but non-penetrating imaging, a clear disposable window may be placed against the tissue to separate the OCT probe from the brain. These probes may or may not need to be catheter based. Catheter-based probes may have a linear scanning movement, similar to the ‘push-pull’ design of LightLab Imaging and probes currently designed for GI endoscopy and dermatology. Non-catheter-based probes may use designs similar to those used for OCT opthalmoscope and OCT microscope. This method is best suited for pathology that is located at a relative short distance from the surface of the tissue. Most spongiform lesions in the cortex are within the detection distance from the surface of the cortex. The present invention also contemplates cutting the sample so that pathology anywhere within the brain may be detected. In such case, the tissue is handled and prepared as for conventional histology.

In the non-contact imaging, a ‘stand-back’ scanning method, which does not require contact with the affected tissue, may also be used. Non-contact imaging provides the least risks for contamination and spread of contagious tissue. In this method, the pathology needs to be close to the surface of the tissue. The tissue may or may not be sliced in preparation.

A characteristic pathology of transmissible spongiform encephalopathies is the presence of widely distributed vacuoles in brain tissue. Imaging these characteristic spongiform changes may serve as a complement to the biochemical assays of the prior art. Optical coherence tomography (OCT), including Fourier-domain OCT (including Spectral domain OCT and Swept-source OCT), is ideally suited to detect these vacuolar changes in brain because they generate high signal contrast. An advantage of OCT diagnosis is that it may be performed in situ, bypassing the need for biopsy. It may also provide answers within seconds or minutes.

While the methods described herein utilize OCT, these are non-limiting examples. Other imaging technologies which allow visualization of vacuoles, back-scattering of vacuoles, vacuole size, or vacuole distribution are also contemplated.

Analyzing vacuole appearance includes visualizing vacuoles, back-scattering of vacuoles, vacuole size, or vacuole distribution. For example, in fresh brain tissue (e.g., not frozen brain tissue, not old brain tissue) detecting the presence of any vacuoles greater than 1-5μ in size by OCT may be presumed pathologic and should be subjected to further studies, such as ELISA. Moreover, vacuoles that are widely distributed, demonstrate a high degree of back scattering, or are large indicate the animal has a transmissible spongiform encephalopathy.

Methods of Diagnosis in Live Animal

The procedures described herein for diagnosis in a slaughtered animal are adaptable for in vivo detection using methods known to the skilled artisan. The least invasive may be to image the olfactory bulb of the animals which is a common site of spongiform changes. A contact or non-contact probe may be placed up the nose of a sedated animal. Minimally invasive procedures include the creation of a burr hole in the skull through which a needle type probe may be inserted. A needle probe may also be inserted directly through the thin roof of the orbital into the frontal cortex. A contact or non-contact probe may also be used if a large enough burr hole is drilled in the skull.

Combination Methods

The present invention also contemplates the use of the methods described herein in combination with other methods of diagnosis. For the diagnosis of BSE, current tests mainly utilize ELISA or Western blots to detect the protease resistant PrP. These tests require biopsy of tissue and typically take hours to complete. Contemplated is the combination of the present methods with these biochemical tests. For example tissue may first be analyzed by the methods described herein. The tissue may then be tested by other methods to confirm the observation.

### EXAMPLES

### Example 1

### CJD, Scrapie, and BSE Diagnosis Using Catheter Based OCT Probe to Visualize Vacuolar Appearance

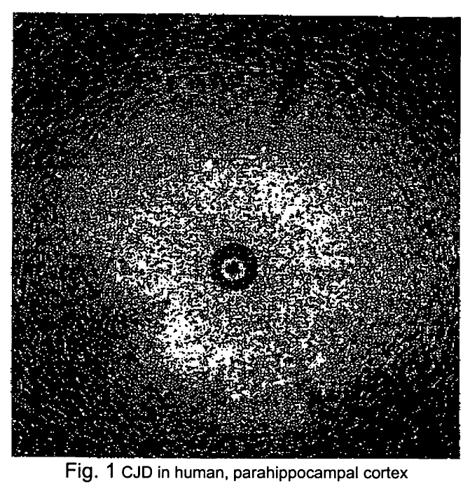

As illustrated in FIG. 1, brain tissue from a patient who died of CJD was imaged using a catheter based OCT probe manufactured by LightLab Imaging (of Westford, Mass.). Large numbers of vacuoles of different diameters were observed. The high degree of back scattering by the vacuoles suggests that they are not simple vacuoles filled with CSF-like fluid. Vacuoles having the observed OCT appearance shown in FIG. 1 have not been observed in human brain stored in the same manner.

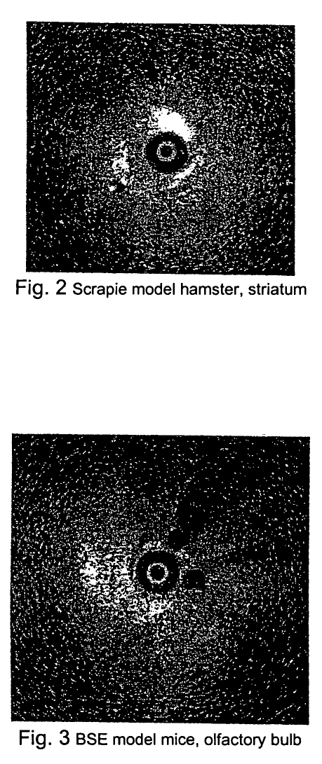

As illustrated in FIG. 2, a hamster infected with scrapie was sacrificed shortly before OCT imaging. Highly reflective vacuoles similar to that observe in CJD brain were observed in the striatum and possibly in the cortex.

As illustrated in FIG. 3, OCT was performed in a mouse brain infected with BSE. Large vacuoles were identified in the olfactory bulb.

### Example 2

### Methods for Screening Tissue of Slaughtered Animals (Imaging Using a Needle-Type Probe)

A catheter-based OCT probe packaged within a rigid cannula (needle-type probe) is inserted into an exposed tissue (i.e. brain, spinal cord, etc) of a slaughtered animal. The approach is used when tissue deep in the brain is desired for sampling and/or testing. A needle type probe may also be inserted directly through thin regions of the skull (i.e. through the roof of orbit below the eye brow to sample the frontal cortex). A radial scan may be performed to image the brain as illustrated in the proceeding figures. The probe will be advanced to sample a volume of tissue. The data may be interpreted by the operator in real time or may be stored for off-line processing. Software may be developed to automatically identify, measure, and count the number of vacuole per volume of tissue sampled. The index of refraction of the vacuole may also be determined based on the amplitude or reflected light. These data will be analyzed using statistical criteria that define the likelihood of TSE in specific brain regions, the animal, and stage of disease.

### Example 3

### Methods for Screening Tissue of a Slaughtered Animal (Contact but Non-Penetrating Imaging)

A clear disposable window may be placed against the tissue to separate the OCT probe from the brain. These probes may or may not need to be catheter based. Catheter-based probes may have a linear scanning movement, similar to the ‘push-pull’ design of LightLab Imaging and probes currently designed for GI endoscopy and dermatology. Non-catheter-based probes may use designs similar to those used for OCT opthalmoscope and OCT microscope. This method is best suited for pathology that is located at a relative short distance from the surface of the tissue. Most spongiform lesions in the cortex are within the detection distance from the surface of the cortex. It is also possible to cut the sample so that pathology anywhere within the brain may be detected. In such case, the tissue would need to be handled but still would not need to be extensively prepared as for conventional histology.

### Example 4

### Methods for Screening Tissue of a Slaughtered Animal (Non-Contact Imaging)

A ‘stand-back’ scanning method that does not require contact with the affected tissue may also be used. Non-contact imaging provides the least risks for contamination and spread of contagious tissue. The limitation is similar to the method described in the preceding paragraph, as the pathology needs to be close to the surface of the tissue. The tissue may or may not be sliced in preparation.

### Example 5

### Methods for in vivo Imaging

The procedures describe for slaughtered animal may be adapted for in vivo detection. The least invasive may be to image the olfactory bulb of the animals which is a common site of spongiform changes. A contact or non-contact probe may be placed up the nose of a sedated animal. Minimally invasive procedures include the creation of a burr hole in the skull through which a needle type probe may be inserted. A needle probe may also be inserted directly through the thin roof of the orbital into the frontal cortex. A contact or non-contact probe may also be used if a large enough burr hole is drilled in the skull.

### REFERENCES

Moynagh Jim, S. H., Kramer, G. N., (1999) The evaluation of Tests for the Diagnosis of Transmissible Spongiform Encephalopathy in Bovines, European Commission, Directorate B—Scientific Health Opinions.

What is claimed is:

1. A method for diagnosing altered neuropathology in an animal, comprising:
(a) imaging brain, spinal cord, or other neural tissue of said animal;
(b) analyzing the appearance of said tissue; and
(c) determining whether the appearance of said tissue is altered relative to corresponding unaltered tissue.

2. The method of claim 1, wherein said neuropathology is altered vacuoles or the presence of plaques.

3. The method of claim 1, wherein said altered neuropathology is selected from the group consisting of transmissible spongiform encephalopathy, bovine spongiform encephalopathy, bovine amyloidotic spongiform encephalopathy, Creutzfield-Jakob disease, scrapie, chronic wasting disease, Gerstmann-Streussler-Sheinker Disease, fatal familial insomnia, hereditary Icelandic syndrome, senility, Alzheimer's disease, and multiple myeloma.

4. The method of claim 1, wherein said imaging occurs via: (1) contact, non-penetrating imaging or (2) non-contact imaging.

5. The method of claim 4, wherein said contact, non-penetrating imaging is optical coherence tomography performed on said tissue, wherein clear material is placed between the tissue to be imaged and the imaging device.

6. The method of claim 5, wherein said optical coherence tomography is performed using a catheter-based probe.

7. The method of claim 5, wherein said optical coherence tomography is performed using a non-catheter-based probe.

8. The method of claim 4, wherein said non-contacting imaging is a “stand-back” scanning method.

9. The method of claim 1, which further comprises (d) confirming said determination regarding the appearance of said tissue using a biochemical test.

10. A method for diagnosing spongiform encephalopathy in an animal, comprising:
(a) imaging brain, spinal cord, or other neural tissue of said animal;
(b) analyzing the appearance of vacuoles in said tissue; and
(c) determining whether the appearance of vacuoles in said tissue is altered relative to corresponding spongiform encephalopathy-free tissue.

11. The method of claim 10, wherein said spongiform encephalopathy is selected from the group consisting of transmissible spongiform encephalopathy, bovine spongiform encephalopathy, bovine amyloidotic spongiform encephalopathy, Creutzfield-Jakob disease, scrapie, and chronic wasting disease.

12. The method of claim 10, wherein said imaging occurs via: (1) contact, non-penetrating imaging or (2) non-contact imaging.

13. The method of claim 12, wherein said imaging is performed using a catheter-based optical coherence tomography probe or a rigid cannula.

14. The method of claim 10, wherein said diagnosis is positive if said vacuoles are widely-distributed, demonstrate a high degree of back scattering of light, or are large.

15. The method of claim 10, wherein said animal is alive and sedated; wherein said tissue is olfactory bulb tissue, thalamus tissue, striatum tissue, or cortex tissue; and wherein said imaging occurs using a probe inserted via a burr-hole drilled in the skull of said animal.

16. The method of claim 10, which further comprises (d) confirming said determination regarding the appearance of said vacuoles using a biochemical test.

17. The method of claim 16, wherein said biochemical test is enzyme-linked immunosorbant assay (ELISA) or Western blot.

18. The method of claim 10, wherein said animal is a bovine, wherein said neural tissue is brain tissue, and wherein said spongiform encephalopathy is bovine spongiform encephalopathy.

19. A method for diagnosing altered neuropathology in an animal, comprising:
(a) step for imaging brain, spinal cord, or other neural tissue of said animal;
(b) step for analyzing the appearance of said tissue; and
(c) step for determining whether the appearance of said tissue is altered relative to corresponding unaltered tissue.

20. A method for diagnosing spongiform encephalopathy in an animal, comprising:
(a) step for imaging brain, spinal cord, or other neural tissue of said animal;
(b) step for analyzing the appearance of vacuoles in said tissue; and
(c) step for determining whether the appearance of vacuoles in said tissue is altered relative to corresponding spongiform encephalopathy-free tissue.

21. An automated method for diagnosing altered neuropathology in an animal, comprising:
(a) automated step for imaging brain, spinal cord, or other neural tissue of said animal;
(b) automated step for analyzing the appearance of said tissue; and
(c) automated step for determining whether the appearance of said tissue is altered relative to corresponding unaltered tissue.

22. An automated method for diagnosing spongiform encephalopathy in an animal, comprising:
(a) automated step for imaging brain, spinal cord, or other neural tissue of said animal;
(b) automated step for analyzing the appearance of vacuoles in said tissue; and
(c) automated step for determining whether the appearance of vacuoles in said tissue is altered relative to corresponding spongiform encephalopathy-free tissue.
